(19) United States
(12) Patent Application Publication
(10) Pub. No.: US 2024/0233881 A1
(43) Pub. Date: Jul. 11, 2024

(54) SYSTEMS AND METHODS FOR DISPERSION OF MOLECULES IN MIXTURES

(71) Applicant: THE BOARD OF REGENTS OF THE UNIVERSITY OF TEXAS SYSTEM, Austin, TX (US)

(72) Inventors: Bruce Beutler, Irving, TX (US); Stephen Lyon, Farmers Branch, TX (US)

(73) Assignee: THE BOARD OF REGENTS OF THE UNIVERSITY OF TEXAS SYSTEM, Austin, TX (US)

(21) Appl. No.: 18/403,608

(22) Filed: Jan. 3, 2024

Related U.S. Application Data
(60) Provisional application No. 63/437,889, filed on Jan. 9, 2023.

(51) Int. Cl. G16C 20/50; G16C 20/20

(52) U.S. Cl. CPC G16C 20/50; G16C 20/20

(57) ABSTRACT

Aspects of the present inventive concept generally relate to systems and methods for dispersion of molecules, and more specifically, for generating layouts indicating assignment of molecules to mixtures. In some aspects, the techniques described herein relate to a method for molecule dispersion, including: generating a first layout of a plurality of molecules in a plurality of mixtures, the plurality of molecules including a set of molecule pairs that appear more than once in the first layout; generating a second layout of the plurality of molecules in the plurality of mixtures, the second layout generated by swapping a first molecule pair of the set of molecule pairs with a second molecule pair of the plurality of molecules; and generating an output layout by comparing a property of the first layout with a property of the second layout.

### CROSS REFERENCE TO RELATED APPLICATION

This application claims priority to U.S. Provisional Patent Application Ser. No. 63/437,889, filed Jan. 9, 2023, and titled “SYSTEMS AND METHODS FOR DISPERSION OF MOLECULES IN MIXTURES,” which is incorporated by reference herein in its entirety.

### BACKGROUND

### 1. Technical Field

Aspects of the present inventive concept generally relate to systems and methods for dispersion of molecules, and more specifically, for generating layouts indicating assignment of molecules to mixtures.

### 2. Discussion of Related Art

Molecules (e.g., antibodies, drugs, or small molecule chemical compounds) may be tested by distributing the molecules into mixtures. Each mixture may include a preset number of molecules so that each mixture can be used in a phenotypic assay. The mixtures may be used to determine a molecule causative of a biological effect.

### SUMMARY

In some aspects, the techniques described herein relate to a method for molecule dispersion, including: generating a first layout of a plurality of molecules in a plurality of mixtures, the plurality of molecules including a set of molecule pairs that appear more than once in the first layout; generating a second layout of the plurality of molecules in the plurality of mixtures, the second layout generated by swapping a first molecule pair of the set of molecule pairs with a second molecule pair of the plurality of molecules; and generating an output layout by comparing a property of the first layout with a property of the second layout.

In some aspects, the techniques described herein relate to an apparatus for molecule dispersion, including: a memory; and one or more processors coupled to the memory, the one or more processors being configured to: generate a first layout of a plurality of molecules in a plurality of mixtures, the plurality of molecules including a set of molecule pairs that appear more than once in the first layout; generate a second layout of the plurality of molecules in the plurality of mixtures, the second layout generated by swapping a first molecule pair of the set of molecule pairs with a second molecule pair of the plurality of molecules; and generate an output layout by comparing a property of the first layout with a property of the second layout.

In some aspects, the techniques described herein relate to a non-transitory computer-readable medium having instructions stored thereon, that when executed by one or more processors, cause the one or more processors to: generate a first layout of a plurality of molecules in a plurality of mixtures, the plurality of molecules including a set of molecule pairs that appear more than once in the first layout; generate a second layout of the plurality of molecules in the plurality of mixtures, the second layout generated by swapping a first molecule pair of the set of molecule pairs with a second molecule pair of the plurality of molecules; and generate an output layout by comparing a property of the first layout with a property of the second layout.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates an example computing device, in accordance with certain aspects of the present inventive concept.

FIG. 2 illustrates the layout associated with these pair counts, in accordance with certain aspects of the present disclosure.

FIG. 3 illustrates techniques for generating an initial layout layer by layer, in accordance with certain aspects of the present disclosure.

FIG. 4 illustrates example operations for molecule dispersion, in accordance with certain aspects of the present inventive concept.

It will be apparent to one skilled in the art after review of the entirety disclosed that the steps illustrated in the figures listed above may be performed in other than the recited order, and that one or more steps illustrated in these figures may be optional.

### DETAILED DESCRIPTION

Certain aspects of the present disclosure are directed to methods and systems for the distribution of molecules into mixtures for testing. For example, certain aspects provide a dispersion system that receives user inputs and provides output data indicating a distribution of molecules in mixtures. The dispersion system may determine the distribution of molecules (e.g., antibodies, drugs, or small molecule chemical compounds) into mixtures in a well format (e.g., 96 wells) in which each well contains a preset number of molecules. Each mixture may be used in a phenotypic assay, and the effects of mixtures can be deconvoluted to determine the molecule causative of a biological effect. The distribution system may determine the proper distribution of molecules in an attempt to maximize the number of times each molecule is tested (e.g., the number of mixtures/wells each molecule is part of) while minimizing the number of mixtures/wells containing the same combination of molecules (e.g., containing the same molecule pairs). This is an attempt to maximize the chances that a molecule with a phenotypic effect can be unambiguously identified from each mixture.

The distribution system may generate a layout in an attempt to reduce the number of errors between different wells and the number of times pairs or triplets are represented in the mixtures. For example, assume 60 antibodies are to be placed into a 96 well plate with 7 randomly chosen antibodies per well. A certain number of ambiguities may exist when assessing the effect of any particular antibody where that antibody would occur with an unrelated antibody that has no biological activity. The antibody may occur three times, five times, or ten times with the unrelated antibody and it may be difficult to ascertain which antibody was responsible for the activity. The dispersion system provided herein increases dispersion and reduces the number of pairs or triplets as compared to conventional implementations (e.g., using a random distribution of molecules in mixtures). In other words, ideally, if a pair appears in one well, it would be preferred if the same pair does not appear in any other well.

In some aspects, the distribution system may receive, as inputs, N which is the number of molecules to be tested for phenotypic effect, M which is the number of mixtures to be made (e.g., typically equal to the number of assays that will be performed), and P which is the preset number of molecules per well/mixture. The distribution system may provide, as output, a template (e.g., layout) showing the identities of molecules to be combined in each mixture.

As an example, for inputs N=60, M=96, and P=7, a layout may be provided that indicates for each of the 96 wells, 7 of the 60 molecules. For instance, the layout may indicate that molecules 1, 26, 27, 36, 41, 55, and 59 are to be included in well 1 (e.g., mixture 1), molecules 4, 11, 15, 20, 37, 41, and 42 are to be included in well 2 (e.g., mixture 2), and so on. The layout may also indicate the wells per molecule. For instance, the layout may indicate that molecule 0 is included in wells 14, 15, 29, 38, 46, 48, 51, 53, 85, 91, 92, and 94.

Some molecules may be present in more wells than other molecules. For example, molecule 0 may be present in 12 wells, while other molecules may be present in 11 wells. Thus, in addition to the layout, the distribution system may indicate the number of times each molecule will be tested, and the number of times any given molecule is co-combined with any other given molecule. The output from the distribution system may be organized to tell, for a given molecule, how many other molecules are co-combined once, twice, three times, four times, etc. with the molecule. The distribution system may also provide a value indicating how likely it is that the degree of dispersion by the template would occur by chance alone. The layout provided by the distribution system may be uploaded to a robotic liquid handling system for automated mixing and may be used in the deconvolution of assay results.

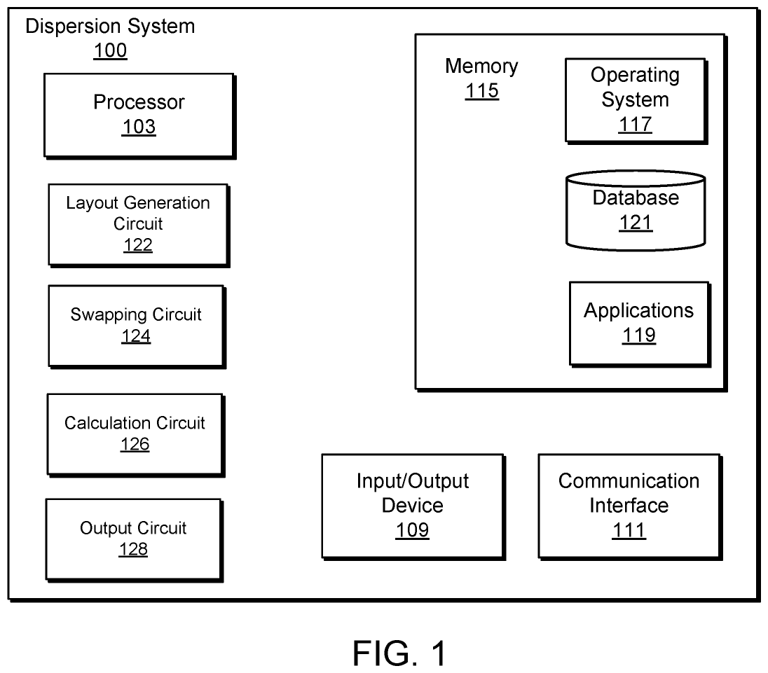

FIG. 1 illustrates an example dispersion system 100, in accordance with certain aspects of the present inventive concept. The dispersion system 100 can include a processor 103 for controlling overall operation of the dispersion system 100 and its associated components, including input/output device 109, communication interface 111, and/or memory 115. A data bus can interconnect processor(s) 103, memory 115, I/O device 109, and/or communication interface 111.

Input/output (I/O) device 109 can include a microphone, keypad, touch screen, and/or stylus through which a user of the dispersion system 100 can provide input and can also include one or more of a speaker for providing audio output and a video display device for providing textual, audiovisual, and/or graphical output. Software can be stored within memory 115 to provide instructions to processor 103 allowing dispersion system 100 to perform various actions. For example, memory 115 can store software used by the dispersion system 100, such as an operating system 117, application programs 119, and/or an associated internal database 121. The various hardware memory units in memory 115 can include volatile and nonvolatile, removable, and non-removable media implemented in any method or technology for storage of information such as computer-readable instructions, data structures, program modules or other data. Memory 115 can include one or more physical persistent memory devices and/or one or more non-persistent memory devices. Memory 115 can include, but is not limited to, random access memory (RAM), read only memory (ROM), electronically erasable programmable read only memory (EEPROM), flash memory or other memory technology, CD-ROM, digital versatile disks (DVD) or other optical disk storage, magnetic cassettes, magnetic tape, magnetic disk storage or other magnetic storage devices, or any other medium that can be used to store the desired information and that can be accessed by processor 103.

Communication interface 111 can include one or more transceivers, digital signal processors, and/or additional circuitry and software for communicating via any network, wired or wireless, using any protocol as described herein. Processor 103 can include a single central processing unit (CPU), which can be a single-core or multi-core processor (e.g., dual-core, quad-core, etc.), or can include multiple CPUs. Processor(s) 103 and associated components can allow the dispersion system 100 to execute a series of computer-readable instructions to perform some or all of the processes described herein. Although not shown in FIG. 1, various elements within memory 115 or other components in dispersion system 100, can include one or more caches, for example, CPU caches used by the processor 103, page caches used by the operating system 117, disk caches of a hard drive, and/or database caches used to cache content from database 121. For implementations including a CPU cache, the CPU cache can be used by one or more processors 103 to reduce memory latency and access time. A processor 103 can retrieve data from or write data to the CPU cache rather than reading/writing to memory 115, which can improve the speed of these operations. In some examples, a database cache can be created in which certain data from a database 121 is cached in a separate smaller database in a memory separate from the database, such as in RAM or on a separate computing device. For instance, in a multi-tiered application, a database cache on an application server can reduce data retrieval and data manipulation time by not needing to communicate over a network with a back-end database server. These types of caches and others can be included in various implementations and can provide potential advantages in certain implementations of software deployment systems, such as faster response times and less dependence on network conditions when transmitting and receiving data.

In certain aspects of the present disclosure, the dispersion system 100 may include a layout generation circuit 122. For example, the layout generation circuit 122 may generate an initial layout to process (e.g., using random assignment of molecules and sequential assignment of molecules). The dispersion system 100 may also include a swapping circuit 124. The swap circuit may swap the assignment of molecules within a particular layout to generate a new layout. The dispersion system 100 may include a calculation circuit 126 that calculates various properties of a layout (e.g., pair counts). The dispersion system 100 may include an output circuit 128, which may output a layout to a liquid handling system for automated mixing.

As described herein, the dispersion system 100 may receive, as program inputs, N (e.g., the number of molecules to be tested for phenotypic effect), M (e.g., the number of mixtures to be made, typically equal to the number of assays that will be performed), and S (e.g., the number of molecules in a single mixture). The dispersion system may also accept a preexisting unoptimized valid layout as a starting point instead of the above parameters. If the above parameters are given as input, the program will initially create a (random) unoptimized layout based on those values and use it as a starting point. As output, the dispersion system may provide, as program output, a layout defined as a list of mixtures with each mixture listing the contained molecules. The output may include statistics on the layout, such as how many times molecule pairs appear together.

Valid layouts may be defined as layouts that have the property that each molecule will appear in T or T+1 mixtures in the layout where:

$$T = floor ( \frac{MS}{N} )$$

so that each molecule is represented as close to the same number of times in the layout as possible. A layout may have certain properties, given the inputs described herein. The number (P) of ways to pair molecules may be represented by equation:

$$P = \frac{N ( N - 1 )}{2}$$

The number of molecule pairs in a single mixture may be represented by expression:

$$\frac{S ( S - 1 )}{2}$$

The number (Q) of paired molecules in the entire set of mixtures may be represented by equation:

$$Q = \frac{MS ( S - 1 )}{2}$$

A property of interest with a layout is the number of times each pair of molecules appear together in a well. That property can be represented as an array (A) of numbers where the index (i) of the array is the number of times a pair appears together and the value (Ai) is the number of such pairs. Thus, the number (P) of ways to pair molecules may be represented by equation:

$$P = {\sum }_{i} {A}_{i}$$

The number (Q) of paired molecules in the entire set of mixtures may be represented by equation:

$$Q = {\sum }_{i} i \cdot {A}_{i}$$

An ideal layout is one where the highest index where the value is non-zero is minimized and the value at that index is minimized. That ideal layout pair array can be calculated. Assuming Q>P, the index:

$$i = floor ( \frac{Q}{P} ) + 1$$

has a number of pair represented by expression:

$$Q = P \times floor ( \frac{Q}{P} )$$

and the remainder is in index I where:

$$i = floor ( \frac{Q}{P} )$$

From a given or calculated random layout, the dispersion system will select a molecule pair at the highest non-zero index and will generate new layouts based on swapping that molecule pair with others, starting with high index pairs. If the new layout is better (lower top index or lower number in same index) it will keep that layout and repeat the process. This continues until the system reaches a layout where all possible swaps have been attempted without improving the layout. That final layout is the output.

As one example, the dispersion system may generate a layout for mixing 60 antibodies in a 96 well plate with 7 antibodies in each well. For this example, N=60, M=96, and S=7. Thus, the number (P) of ways to pair molecules is 1770 and the number (Q) of paired molecules in the entire set of mixtures is 2016. Thus, an ideal layout would have 246 antibody pairs appearing twice together (e.g., since the number of paired molecules in the entire set of mixtures (2016) minus the number of ways to pair the molecules (1770) is equal to 246). Moreover, the ideal layout would have 1524 antibody pairs appearing once time together (e.g., since the number of ways to pair the molecules (1770) minus the 246 antibody pairs appearing twice together is equal to 1524). This can be represented as an array:
Ideal layout pair counts=[0, 1524, 246, 0, 0, 0, 0, 0, 0, 0, 0, 0, 0]
   Where A₀ equals 0 and represents the number of antibody pairs that do not appear together, A₁ is 1524 and represents the number of antibody pairs that appear once together, and A₂ is 246 and represents the number of antibody pairs that appear twice together. The highest index is T+1, which is 12 in this example. While the dispersion system may generate a layout that has ideal pair counts, the dispersion system may generate a less-than-ideal layout in some scenarios (e.g., due to limitations in computation resources, time, or the initial layout used). After multiple runs starting from random layouts, the dispersion system may generate a layout having the following pair counts:
Generated layout pair counts=[113, 1298, 359, 0, 0, 0, 0, 0, 0, 0, 0, 0, 0]

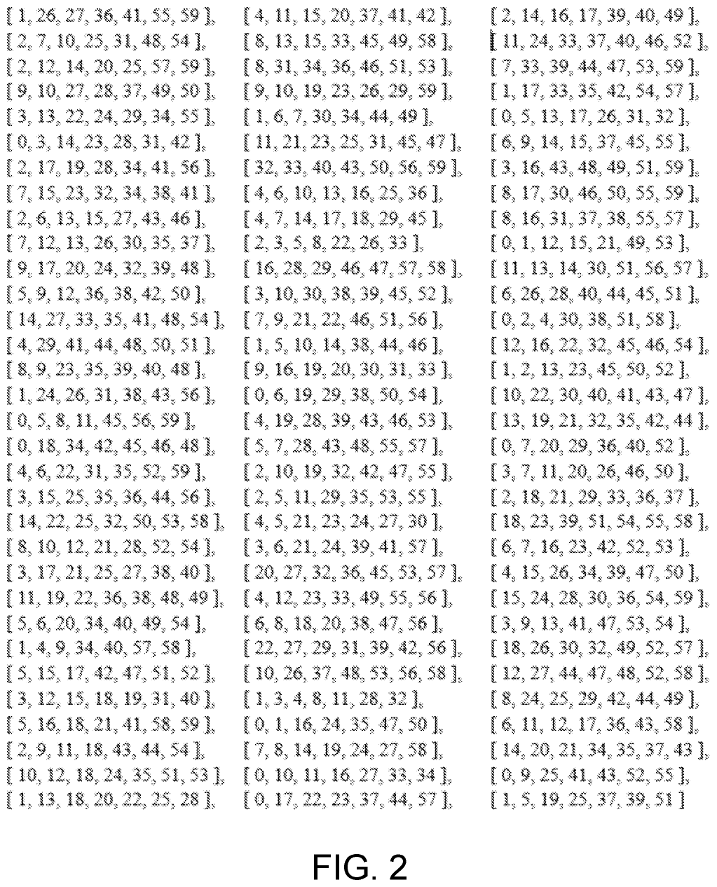

FIG. 2 illustrates the layout associated with pair counts [113, 1298, 359, 0, 0, 0, 0, 0, 0, 0, 0, 0, 0], in accordance with certain aspects of the present disclosure. The layout is represented by 96 arrays (e.g., each indicating the molecules to be included in each of the 96 mixtures) of length 7, where the antibodies are numbered 0-59.

To generate the layout, an initial layout (e.g., a first layout) is first generated. The initial layout may be generated in any suitable manner. For example, the initial layout may be generated by randomly assigning the molecules to mixtures, while still avoiding any one of the molecules to repeat in any one of the mixtures.

Once the first layout is generated, the pair counts for the layout are identified. A group of pairs that repeat in the mixture the greatest number of times may be identified. For example, for generated layout pair counts [113, 1298, 359, 0, 0, 0, 0, 0, 0, 0, 0, 0, 0], 359 molecule pairs repeat the greatest number of times (e.g., twice) since no other molecule pairs repeat more than two times. One or more of molecule pairs in the group of pairs that repeat the greatest number of times may be swapped from one mixture in the initial layout to another mixture. For instance, assuming a first molecule pair (e.g., molecules 41 and 55) in a first mixture and a second molecule pair (e.g., molecules 12 and 36) in a second mixture repeat the greatest number of times in the mixtures, the first and second molecule pairs may be swapped. In other words, the second molecular pair may be assigned to the first mixture and the first molecular pair may be assigned to the second mixture, in effect generating a second layout.

The pair counts for the second layout may be calculated and compared to the pair counts of the first layout. Assuming the second layout has improved pair counts (e.g., the group of pairs that repeat the greatest number of times in the second layout is less than the group of pairs that repeat the greatest number of times in the first layout), the second layout may be considered as the output layout. This process may repeat multiple times. For example, molecule pairs in the second layout may be swapped using a similar process to generate a third layout, and pair counts may be calculated and compared, and so on. This process may continue until new layouts being generated are not showing improvements over old layouts. In some aspects, the process may be repeated multiple times using different initial layouts since using one initial layout may result in the dispersion system generating a more improved layout as compared to using a different initial layout.

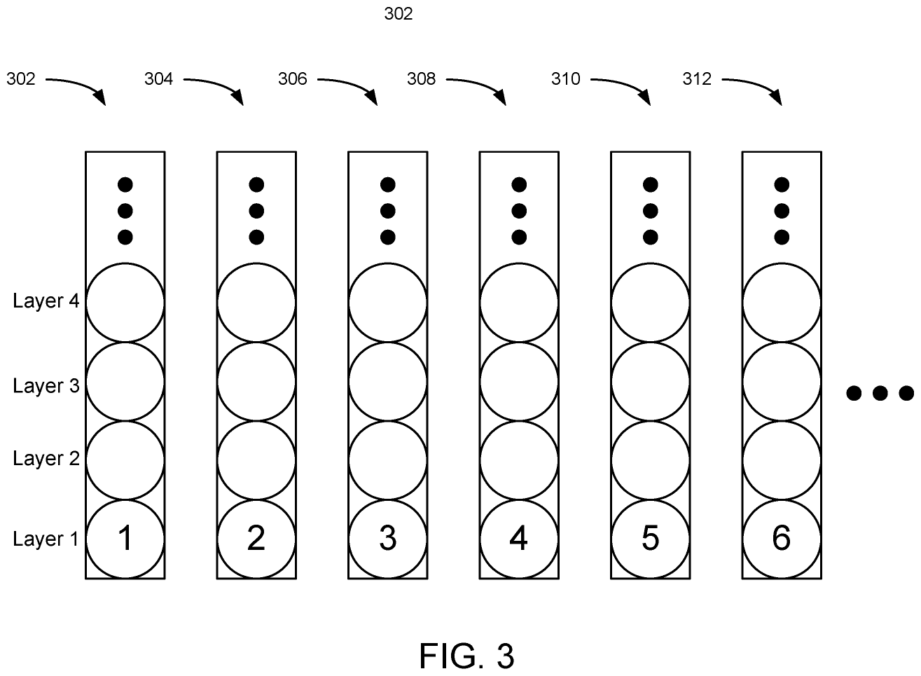

FIG. 3 illustrates techniques for generating an initial layout layer by layer, in accordance with certain aspects of the present disclosure. As shown, there may be a number of mixtures 302, 304, 306, 308, 310, 312. While six mixtures are shown, any number of mixtures may be used. To generate an initial layout for the dispersion system, a first layer (layer 1) of the mixtures may be assigned molecules in sequential order, followed by the second layer (layer 2), and so on. The mixtures may be filled layer by layer and sequentially while avoiding any mixture to have more than one of a particular molecule.

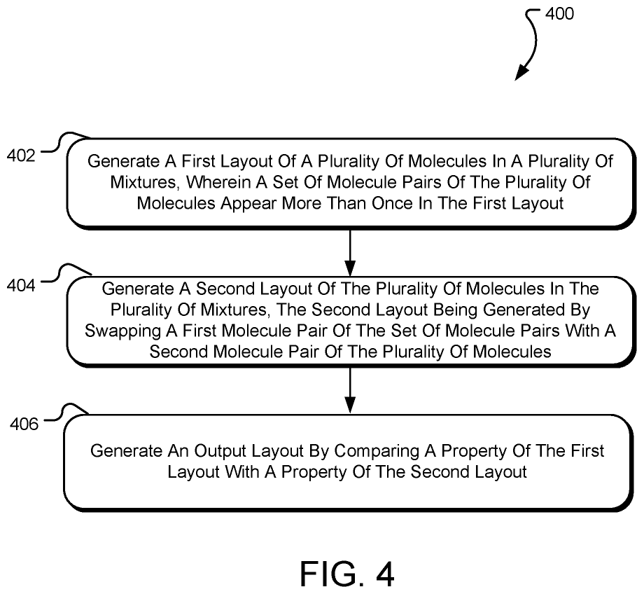

FIG. 4 illustrates example operations 400 for molecule dispersion, in accordance with certain aspects of the present inventive concept. The operations 400 may be performed, for example, by a dispersion system, such as the dispersion system 100 of FIG. 1.

At block 402, the dispersion system generates a first layout of a plurality of molecules in a plurality of mixtures. A set of molecule pairs of the plurality of molecules may appear more than once in the first layout.

At block 404, the dispersion system generates a second layout of the plurality of molecules in the plurality of mixtures. The second layout may be generated by swapping a first molecule pair of the set of molecule pairs with a second molecule pair of the plurality of molecules. In the first layout or the second layout, any one of the plurality of molecules does not repeat in any one of the plurality of mixtures. At block 406, the dispersion system generates an output layout by comparing a property (e.g., pair counts) of the first layout with a property (e.g., pair counts) of the second layout.

In some aspects, the dispersion system identifies the first molecule pair based on the first molecule pair being part of a first group of molecule pairs appearing a greatest quantity of times in the first layout. In some aspects, generating the output layout may include selecting, as the output layout, one of the first layout and the second layout based on the comparison. For example, selecting one of the first layout and the second layout may include selecting the second layout based on a second group of molecule pairs appearing a greatest quantity of times in the second layout being less than the first group of molecule pairs.

In some aspects, the dispersion system receives a first user input indicating a quantity of the plurality of molecules, a second input indicating a quantity of the plurality of mixtures, and a third input indicating a quantity of molecules in each of the plurality of mixtures. The first layout and the second layout may be generated based on the first input, the second input, and the third input.

In some aspects, the output layout is one of multiple output layouts generated starting with different initial layouts. For example, at least one of the different initial layouts may be generated by randomly assigning each of the plurality of molecules to each of the plurality of mixtures. In some aspects, a first one of the different initial layouts may be generated by sequentially assigning the plurality of molecules to a first layer of the plurality of mixtures and to a second layer of the plurality of mixtures after the first layer has been filled.

The technology disclosed herein can be used to find mixtures for multiplex screening of any type of molecule in biological assays. Examples of screens may include a monoclonal antibody screen (e.g., which could be performed in vivo either by administering purified antibodies or by administering hybridomas or other cells secreting these monoclonal antibodies). Other examples may include a synthetic compound screen, a screen of cytotoxic T cell clones, a peptide screen, a screen of proteins, a screen of lipids, a screen of carbohydrates, a screen of fractionated biological materials of any source, or for pharmacological effects in mice (e.g., in treating cancer or any other type of disease) or cells (e.g., inducing cytokine production or any other measurable cellular event), or for binding to target protein(s) or cell(s).

In some aspects, to measure a quality of a layout (e.g., to determine if one layout is improved over another), the dispersion system may assign a score to the layout as the number of those pairs with counts equal to or above the maximum non-zero index count in the ideal layout, times the index, in accordance with the following equation:

$$( {A}_{( P \cdot ⌊ \frac{Q}{P} ⌋ + 1} - Q + P \cdot ⌊ \frac{Q}{P} ⌋ ) \cdot ( P \cdot ⌊ \frac{Q}{P} ⌋ + 1 ) + {\sum }_{i = ⌊ \frac{Q}{P} ⌋ + 2}^{T + 1} i \cdot {A}_{i}$$

The ideal layout described herein may have a score of 0. The layout having pair counts [113, 1298, 359, 0, 0, 0, 0, 0, 0, 0, 0, 0, 0] has a score of 226. Using this metric on a set of 1092 randomly generated layouts (using N=60, M=96, and S=7) gives a mean score of 834.7 and a standard deviation of 21.5. Compared to random layouts, the layouts generated by the dispersion system have a score over 28 standard deviations from the mean. The dispersion system provides a practical method of generating mixing layouts that will aid in deconvoluting assay results to identify causative molecules for phenotypes. Improvements and/or repeated runs have the potential to bring the generated layout of the dispersion system closer to an ideal layout.

These and various other arrangements will be described more fully herein. As will be appreciated by one of skill in the art upon reading the following disclosure, various aspects described herein can be a method, a computer system, or a computer program product. Accordingly, those aspects can take the form of an entirely hardware implementation, an entirely software implementation, or at least one implementation combining software and hardware aspects. Furthermore, such aspects can take the form of a computer program product stored by one or more computer-readable storage media (e.g., non-transitory computer-readable medium) having computer-readable program code, or instructions, included in or on the storage media. Any suitable computer-readable storage media can be utilized, including hard disks, CD-ROMs, optical storage devices, magnetic storage devices, and/or any combination thereof. In addition, various signals representing data or events as described herein can be transferred between a source and a destination in the form of electromagnetic waves traveling through signal-conducting media such as metal wires, optical fibers, and/or wireless transmission media (e.g., air and/or space).

Implementations of the present inventive concept include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

While specific implementations are discussed, it should be understood that this is done for illustration purposes only. A person skilled in the relevant art will recognize that other components and configurations may be used without parting from the spirit and scope of the present inventive concept. Thus, the following description and drawings are illustrative and are not to be construed as limiting. Numerous specific details are described to provide a thorough understanding of the present inventive concept. However, in certain instances, well-known or conventional details are not described in order to avoid obscuring the description. References to one or an implementation in the present inventive concept can be references to the same implementation or any implementation; and such references mean at least one of the implementations.

Reference to “one implementation” or “an implementation” means that a particular feature, structure, or characteristic described in connection with the implementation is included in at least one implementation of the present inventive concept. The appearances of the phrase “in one implementation” in various places in the specification are not necessarily all referring to the same implementation, nor are separate or alternative implementations mutually exclusive of other implementations. Moreover, various features are described which may be exhibited by some implementations and not by others.

The terms used in this specification generally have their ordinary meanings in the art, within the context of the present inventive concept, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the present inventive concept or of any example term. Likewise, the present inventive concept is not limited to various implementations given in this specification.

Without intent to limit the scope of the present inventive concept, examples of instruments, apparatus, methods and their related results according to the implementations of the present inventive concept are given below. Note that titles or subtitles may be used in the examples for convenience of a reader, which in no way should limit the scope of the present inventive concept. Unless otherwise defined, technical and scientific terms used herein have the meaning as commonly understood by one of ordinary skill in the art to which the present inventive concept pertains. In the case of conflict, the present document, including definitions will control.

Additional features and advantages of the present inventive concept will be set forth in the description which follows, and in part will be obvious from the description, or can be learned by practice of the herein disclosed principles. The features and advantages of the present inventive concept can be realized and obtained by means of the instruments and combinations particularly pointed out in the appended claims. These and other features of the present inventive concept will become more fully apparent from the following description and appended claims or can be learned by the practice of the principles set forth herein.

What is claimed is:

1. A method for molecule dispersion, comprising:
generating a first layout of a plurality of molecules in a plurality of mixtures, the plurality of molecules including a set of molecule pairs that appear more than once in the first layout;
generating a second layout of the plurality of molecules in the plurality of mixtures, the second layout generated by swapping a first molecule pair of the set of molecule pairs with a second molecule pair of the plurality of molecules; and
generating an output layout by comparing a property of the first layout with a property of the second layout.

2. The method of claim 1, further comprising:
identifying the first molecule pair based on the first molecule pair being part of a first group of molecule pairs appearing a greatest quantity of times in the first layout.

3. The method of claim 2, wherein the generating of the output layout includes selecting, as the output layout, one of the first layout and the second layout based on the comparison.

4. The method of claim 3, wherein the selecting of the one of the first layout and the second layout includes selecting the second layout based on a second group of molecule pairs appearing a greatest quantity of times in the second layout being less than the first group of molecule pairs.

5. The method of claim 1, further comprising:
receiving a first user input indicating a quantity of the plurality of molecules, a second input indicating a quantity of the plurality of mixtures, and a third input indicating a quantity of molecules in each of the plurality of mixtures, the first layout and the second layout generated based on the first input, the second input, and the third input.

6. The method of claim 1, wherein, in the first layout or the second layout, any one of the plurality of molecules does not repeat in any one of the plurality of mixtures.

7. The method of claim 1, wherein the output layout is one of multiple output layouts generated starting with different initial layouts.

8. The method of claim 7, wherein at least one of the different initial layouts is generated by randomly assigning each of the plurality of molecules to each of the plurality of mixtures.

9. The method of claim 7, wherein the different initial layouts includes a first one that is generated by sequentially assigning the plurality of molecules to a first layer of the plurality of mixtures and to a second layer of the plurality of mixtures after the first layer has been filled.

10. An apparatus for molecule dispersion, comprising:
a memory; and
one or more processors coupled to the memory, the one or more processors being configured to:
generate a first layout of a plurality of molecules in a plurality of mixtures, the plurality of molecules including a set of molecule pairs that appear more than once in the first layout;
generate a second layout of the plurality of molecules in the plurality of mixtures, the second layout generated by swapping a first molecule pair of the set of molecule pairs with a second molecule pair of the plurality of molecules; and
generate an output layout by comparing a property of the first layout with a property of the second layout.

11. The apparatus of claim 10, wherein the one or more processors are further configured to identify the first molecule pair based on the first molecule pair being part of a first group of molecule pairs appearing a greatest quantity of times in the first layout.

12. The apparatus of claim 11, wherein, to generate the output layout, the one or more processors are configured to select, as the output layout, one of the first layout and the second layout based on the comparison.

13. The apparatus of claim 12, wherein, to select one of the first layout and the second layout, the one or more processors are configured to select the second layout based on a second group of molecule pairs appearing a greatest quantity of times in the second layout being less than the first group of molecule pairs.

14. The apparatus of claim 10,
wherein,
the one or more processors are further configured to receive a first user input indicating a quantity of the plurality of molecules, a second input indicating a quantity of the plurality of mixtures, and a third input indicating a quantity of molecules in each of the plurality of mixtures, and
the first layout and the second layout are generated based on the first input, the second input, and the third input.

15. The apparatus of claim 10, wherein, in the first layout or the second layout, any one of the plurality of molecules does not repeat in any one of the plurality of mixtures.

16. The apparatus of claim 10, wherein the output layout is one of multiple output layouts generated starting with different initial layouts.

17. The apparatus of claim 16, wherein at least one of the different initial layouts is generated by randomly assigning each of the plurality of molecules to each of the plurality of mixtures.

18. The apparatus of claim 16, wherein a first one of the different initial layouts is generated by sequentially assigning the plurality of molecules to a first layer of the plurality of mixtures and to a second layer of the plurality of mixtures after the first layer has been filled.

19. A non-transitory computer-readable medium having instructions stored thereon, that when executed by one or more processors, cause the one or more processors to:
generate a first layout of a plurality of molecules in a plurality of mixtures, the plurality of molecules including a set of molecule pairs that appear more than once in the first layout;
generate a second layout of the plurality of molecules in the plurality of mixtures, the second layout generated by swapping a first molecule pair of the set of molecule pairs with a second molecule pair of the plurality of molecules; and
generate an output layout by comparing a property of the first layout with a property of the second layout.

20. The non-transitory computer-readable medium of claim 19, wherein the instructions further cause the one or more processors to identify the first molecule pair based on the first molecule pair being part of a first group of molecule pairs appearing a greatest quantity of times in the first layout.
